(19) United States
(12) Patent Application Publication
(10) Pub. No.: US 2020/0253173 A1
(43) Pub. Date: Aug. 13, 2020

(54) FISH AND METHOD FOR PRODUCING FISH

(71) Applicants: KYOTO UNIVERSITY, Kyoto-shi, Kyoto (JP); KINKI UNIVERSITY, Higashiosaka-shi, Osaka (JP)

(72) Inventors: Masato KINOSHITA, Kyoto (JP); Yasutoshi YOSHIURA, Kagawa (JP); Hiroyuki YOSHIKAWA, Yamaguchi (JP); Keitaro KATO, Wakayama (JP)

(21) Appl. No.: 16/651,862

(22) PCT Filed: Sep. 28, 2018
(86) PCT No.: PCT/JP2018/036522

§ 371 Date: Mar. 27, 2020

(30) Foreign Application Priority Data
Sep. 28, 2017 (JP) 2017-187374

(51) Int. Cl. A01K 67/027

(52) U.S. Cl. CPC A01K 67/0276; A01K 2227/40; A01K 2217/054

(57) ABSTRACT

The present invention provides fish with promoted growth. The fish according to the present invention has loss of function of a melanocortin-4 receptor (MC4R) gene.

### TECHNICAL FIELD

The present invention relates to fish and a method for producing fish.

### BACKGROUND ART

Some fish such as Takifugu rubripes are supplied through aquaculture. However, the growth of Takifugu rubripes by aquaculture takes a long time, for example, two years in males and three years in females until sexually mature adult fish are obtained. For this reason, shortening of a fish breeding period is required for aquaculture of the fish (Non Patent Literature 1).

Aquacultured adult fish such as of Takifugu rubripes are smaller than natural adult fish. For this reason, an increase in yield (weight) of the aquacultured adult fish is required.

### CITATION LIST

### Non Patent Literature

Non Patent Literature 1: Shao Jun Du et al., “Growth enhancement in transgenic atlantic salmon by the use of an “all fish” chimeric growth hormone gene construct”, 1992, Biotechnology, Vol. 10, pages 176-181

### SUMMARY OF INVENTION

### Technical Problem

Hence, the present invention is intended to provide fish with promoted growth.

### Solution to Problem

In order to achieve the aforementioned object, the fish according to the present invention has loss of function of a melanocortin-4 receptor (MC4R) gene.

The method for producing fish according to the present invention (hereinafter also referred to as the “production method”) includes interbreeding the fish according to the present invention with another fish.

### Advantageous Effects of Invention

The present invention can provide, for example, fish with promoted growth compared to the fish having a normal-type MC4R gene.

### BRIEF DESCRIPTION OF DRAWINGS

FIG. 1 is a graph showing the body weight of Takifugu rubripes with loss of function of an MC4R gene in Example 1.

FIG. 2 is a graph showing the body weight of Takifugu niphobles with loss of function of an MC4R gene in Example 2.

FIG. 3 is a graph showing the body weight of Oryzias latipes with loss of function of an MC4R gene in Example 4.

FIG. 4 is a graph showing the feed intake of Oryzias latipes with loss of function of an MC4R gene in Example 5.

### DESCRIPTION OF EMBODIMENTS

The fish according to the present invention has, for example, a partial or complete deletion of an MC4R gene.

The fish according to the present invention belongs to at least one selected from the group consisting of the family Tetraodontidae, the family Sparidae, the family Serranidae, and the family Adrianichthyidae, for example.

The fish according to the present invention is, for example, suitable for aquaculture.

The growth of the fish according to the present invention is promoted compared to, for example, control fish having a normal MC4R gene.

The fish according to the present invention is, for example, an edible portion of the fish.

The production method according to the present invention further includes, for example, growing the fish obtained by the interbreeding.

The production method according to the present invention further includes, for example, prior to the interbreeding, measuring an expression level of melanocortin-4 receptor (MC4R) in a biological sample of test fish; and selecting the fish according to the present invention from the test fish, and in the selecting, fish with loss of function of the MC4R gene is selected on the basis of the expression level of the MC4R in the biological sample of the test fish and a reference value.

The production method according to the present invention further includes, prior to the interbreeding, selecting the fish according to the present invention from the test fish, for example.

The production method according to the present invention includes, for example, measuring an expression level of melanocortin-4 receptor (MC4R) in a biological sample of test fish; and in the selecting, fish with loss of function of the MC4R gene is selected on the basis of the expression level of the MC4R in the biological sample of the test fish and a reference value.

The production method according to the present invention further includes, for example, prior to the interbreeding, creating the fish according to the present invention from target fish, and the creating includes mutating the MC4R gene of the target fish to have a loss-of-function mutation.

In the production method according to the present invention, the mutation of the MC4R gene to have loss of function is, for example, a partial or complete deletion mutation of the MC4R gene.

In the production method according to the present invention, the mutating includes, for example, a mutation step of mutating an MC4R gene of the target fish, and a mutation selection step of selecting the target fish with a loss of function mutation of the MC4R gene from the target fish obtained in the mutation step.

The production method according to the present invention further includes, for example, a measuring step of measuring an expression level of the MC4R in a biological sample of the target fish after the mutation step, and in the mutation selection step, the target fish with loss of function mutation of the MC4R gene is selected on the basis of the expression level of MC4R in the biological sample of the target fish and a reference value.

In the production method according to the present invention, the expression level of the MC4R is, for example, the expression level of the protein of the MC4R gene.

In the production method according to the present invention, the biological sample is, for example, the brain.

<Fish>

The fish according to the present invention has loss of function of a melanocortin-4 receptor (MC4R) gene as mentioned above. The fish according to the present invention is characterized by having loss of function of the MC4R gene, and other aspects and conditions are not particularly limited. The fish according to the present invention can be described with reference to the descriptions of a production method, a screening method, a growth promotion method, and a mutated MC4R gene according to the present invention, to be described later, for example.

As a result of earnest studies, the inventors of the present invention found that the MC4R gene is associated with the growth of the fish, specifically, the loss of function of the MC4R gene promotes the growth of the fish, and established the present invention. The fish according to the present invention has loss of function of the MC4R gene, and thus has a normal-type MC4R gene (hereinafter also referred to as “normal MC4R gene”) and is grown more promptly than fish having the same gene except for the MC4R gene (hereinafter also referred to as “wild-type fish” or “control fish”), for example. The growth may include, for example, elongation of the body length, the increase in body weight, and the increase in feed intake of the fish. The fish according to the present invention allows, for example, the breeding period until the fish reaches the target growth stage to be shortened as compared to the wild-type fish. Thus, the fish according to the present invention is suitable for use as fish for aquaculture. Although the mechanism is unknown, the period of time required for the fish according to the present invention to be grown as sexually mature fish is shorter than that of the wild-type, for example. For this reason, the fish according to the present invention can be interbred (for example, egg collection, sperm collection) in a shorter period of time, as compared to the wild-type fish, for example. Thus, the fish according to the present invention is suitable for use as fish for aquaculture.

In the present invention, the “fish” means, for example, animals classified into an animal group excluding tetrapods from animals in vertebrata subphylum. Specific examples of the fish include fish belonging to the family Tetraodontidae such as puffers, fish belonging to the family Ostraciidae such as boxfishes, fish belonging to the family Sparidae such as sea breams and porgies, fish belonging to the family Serranidae such as sea basses, fish belonging to the family Oryziidae such as medakas, and the fish belonging to the family Paralichthys. Examples of the fish belonging to the family Tetraodontidae include fish belonging to the genus Takifugu such as Takifugu rubripes, Takifugu porphyreus, and Takifugu niphobles and fish belonging to genus Lagocephalus such as Lagocephalus wheeleri. Examples of the fish belonging to the family Ostraciidae include fish belonging to the genus Ostracion such as Ostracion immaculatus. Examples of the fish belonging to the family Sparidae include fish belonging to the genus Pagrus such as Pagrus major and Pagrus auratus, fish belonging to the genus Acanthopagrus such as Acanthopagrus schlegelii and Acanthopagrus latus, and fish belonging to the genus Dentex such as Dentex tumifrons. Examples of the fish belonging to the family Serranidae include fish belonging to the genus Epinephelus such as Epinephelus septemfasciatus, Epinephelus bruneus, Epinephelus akaara, and Epinephelus malabaricus and fish belonging to the genus Plectropomus such as Plectropomus leopardus. Examples of the fish belonging to the family Oryziidae include fish belonging to the genus Oryzias such as Medaka (Oryzias latipes, Oryzias sakaizumii) and Oryzias javanicus. The fish belonging to the family Paralichthys can be, for example, Paralichthys olivaceus. In the present invention, the fish is, for example, fish for aquaculture in a preferred embodiment.

In the present invention, the fish may be the whole or a part (portion) of the fish. If the fish is whole fish, the growth stage of the fish is not particularly limited, and may be, for example, any of larvae, juveniles, immature fish (fingerling fish, young fish), and adult fish. The part of the fish is not particularly limited and can be, for example, an edible portion of the fish. Examples of the edible portion include a muscle, an esophagus, a stomach, a pyloric appendage, an intestinal tract, a testis, an ovary, a liver, a spleen, a heart, a floating bag, and an epidermis.

In the present invention, a melanocortin-4 receptor (MC4R) gene may be an MC4R gene of fish. Specific examples of the MC4R gene of the fish include the MC4R genes (normal MC4R gene) in Table 1 below. The accession numbers in Table 1 below are the accession numbers in the GenBank.

TABLE 1

| | mRNA of MC4R gene | MC4R protein |
|---|---|---|
| Takifugu rubripes | Accession No. AB437814 | Accession No.: BAG38469 |
| | (SEQ ID NO: 1) | |
| Pagrus major | SEQ ID NO: 2 | — |
| Takifugu niphobles | SEQ ID NO: 3 | — |
| Oryzias latipes | Accession No. XM_004081195 | Accession No.: XP_004081243 |
| | (SEQ ID NO: 4) | |
| Danio rerio | Accession No. NM_173278 | Accession No.: NP_775385 |
| Callorhinchus milii | Accession No. XM_007895520 | Accession No.: XP_007893711 |
| Paralichthys olivaceus | Accession No.: XM_020105498 | Accession No.: XP_019961057 |
| Poecilia reticulata | Accession No.: XM_008396409 | Accession No.: XP_008394631 |

mRNA of MC4R gene derived from Takifugu rubripes
(SEQ ID NO: 1)
5′-ATGAACGCCACCGATCCCCCTGGGAGGGTGCAGGACTTCAGCAACG
GGAGCCAAACCCCGGAGACGGACTTTCCAAACGAGGAGAAGGAATCGTC
TACGGGATGCTACGAGCAGATGCTGATCTCCACGGAGGTGTTCCTGACT
CTGGGAATCATCAGCCTGCTGGAGAACATCCTGGTGGTCGCCGCTATAG
TGAAGAACAAGAATCTCCACTCGCCCATGTACTTTTTCATCTGCAGCCT
GGCCGTGGCCGACATGCTCGTGAGCGTCTCCAACGCCTCCGAGACGATC
GTCATAGCGCTCATCAACAGCGGCACGCTGACCATCCCCGCCACGCTGA
TCAAGAGCATGGACAACGTGTTTGACTCCATGATCTGCAGCTCTTTGCT
GGCGTCCATCTGCAGCCTGCTCGCCATCGCCGTCGACCGCTACATCACC
ATCTTCTACGCCCTGCGCTACCACAACATCGTCACCCTGCGGAGAGCCT
CGCTGGTCATCAGCAGCATCTGGACGTGCTGCACCGTGTCCGGCGTGCT
CTTCATCGTCTACTCGGAGAGCACCACCGTGCTCATCTGCCTCATCACC
ATGTTCTTCACCATGCTGGTGCTCATGGCCTCCCTCTACGTCCACATGT
TCCTGCTGGCGCGCCTGCACATGAAGCGGATCGCGGCGATGCCGGGCAA
CGCGCCCATCCACCAGAGAGCCAACCTGAAGGGCGCCATCACCCTCACC
ATCCTCCTGGGAGTGTTTGTGGTCTGCTGGGCGCCTTTCTTCCTTCACC
TCATCCTCATGATCACCTGCCCCAAGAACCCATACTGCACGTGCTTCAT
GTCCCACTTCAACATGTACCTCATCCTCATCATGTGCAACTCCGTCATC
GACCCCATCATCTACGCCTTTCGCAGCCAGGAGATGAGAAAAACCTTCA
AGGAGATCTTCTGCTGCTCCCAAATGCTGGTGTGCATGTGA-3′
mRNA of MC4R gene derived from Pagrus major
(SEQ ID NO: 2)
5′-ATGAACAGCACAGATCTCCATGGATTGATCCAGGGCTACCACAACA
GGAGCCAAACGTCAGTTTTGCCTCTGAACAAAGACTTACCAGCCGAGGA
GAAGGACTCATCGGCAGGATGCTACGAACAGCTGCTGATTTCTACAGAG
GTGTTCCTCACTCTGGGCATCATCAGCCTGCTGGAGAACATCCTGGTTG
TTGCTGCAATCGTCAAGAACAAGAACCTTCACTCGCCCATGTACTTCTT
CATCTGTAGCCTCGCTGTTGCTGACATGCTCGTGAGCGTCTCCAACGCC
TCCGAGACCATCGTCATAGCGCTCATCGATGGAGGCAACCTGACCATCC
CCGCCACGCTGATCAAGAACATGGACAATGTATTTGACTCTATGATCTG
TAGCTCTCTGTTAGCGTCTATCTGCAGCTTGCTCGCCATCGCCATCGAT
CGCTACATCACCATCTTCTACGCGCTGCGGTACCACAACATTGTCACCC
TGCGGAGAGCCATATTGGTCATCAGCAGCATCTGGACGTGCTGCACCGT
CTCTGGCATCCTCTTCATCATCTACTCAGAGAGCACCACGGTGCTCATC
TGCCTCATCACCATGTTCTTCACCATGCTCGTTCTCATGGCGTCGCTCT
ACGTGCACATGTTCCTTCTGGCGCGCTTGCACATGAAGCGGATCGCCGC
TCTGCCGGGCAACGCGCCCATCCACCAGCGGGCCAACATGAAGGGCGCC
ATCACCCTCACCATCCTCCTCGGGGTGTTCGTGGTGTGCTGGGCGCCCT
TCTTCCTCCACCTCATCCTCATGATCACCTGCCCCAGGAACCCCTACTG
CACCTGCTTCATGTCCCACTTCAACATGTACCTCATCCTCATCATGTGC
AACTCCGTCATCGACCCCATCATCTACGCTTTCCGCAGCCAGGAGATGA
GGAAGACCTTCAAGGAGATTTTCTGCTGCTCTCACACTTTCCTGTGCGT
Gtga-3′
Partial sequence of mRNA of MC4R gene derived
from Takifugu niphobles
(SEQ ID NO: 3)
5′-GGAGGTGTTCCTGACTCTGGGAATCATCAGCCTGCTGGAGAACATC
CTGGTGGTCGCCGCTATAGTGAAGAACAAGAATCTCCACTCGCCCATGT
ACTTTTTCATCTGCAGCCTGGCCGTGGCCGACATGCTCGTGAGCGTCTC
CAACGCCTCCGAGACGATCGTCATAGCGCTCATCAACAGCGGCACGCTC
ACCATCCCCGCCACGCTGATCAAGAGCATGGACAACGTGTTTGACTCCA
TGATCTGCAGCTCCTTGCTGGCGTCCATCTGCAGCCTGCTCGCCATCGC
CGTCGACCGCTACATCACCATCTTCTACGCCCTGCGCTACCACAACATC
GTCACCCTGCGGAGAGCCTCGCTGGTCATCAGCAGCATCTGGACGTGCT
GCACCGTGTCCGGCGTGCTCTTCATCGTCTACTCGGAGAGCACCACCGT
GCTCATCTGCCTCATCACCATGTTCTTCACCATGCTGGTGCTCATGGCC
TCCCTCTACGTCCACATGTTCCTGCTGGCGCGCCTGCACATGAAGCGGA
TCGCGGCGATGCCGGGCAACGCGCCCATCCACCAGAGAGCCAACCTGAA
GGGCGCCATCACCCTCACCATCCTCCTGGGAGTGTTTGTGGTCTGCTGG
GCGCCTTTCTTCCTTCACCTCATCCTCATGATCACCTGCCCCAAGAACC
CATACTGCACGTGCTTCATGTCCCACTTCAACATGTACCTCATCCTCAT
CATGTGCAACTCCGTCATCGACCCCATCATCTACGCCTTTCGCAGCCAG
GAGATGA-3′
mRNA of MC4R gene derived from Oryzias latipes
(SEQ ID NO: 4)
5′-ATGAACTCCACTCTGCCTTATGGGTCGGTCCCCAACAGAAGCCTCT
CCTCGGCCACTCTCCCTCCTGACCTGGGAGGACAGAAAGACTCGTCGGC
GGGATGCTACGAGCAGCTTCTGATCTCCACTGAGGTCTTCCTCACTTTG
GGCATCATCAGCCTGCTGGAGAACATCCTGGTTGTTGCTGCGATCGTTA
AAAACAAGAACCTCCACTCCCCCATGTACTTTTTCATCTGCAGCCTCGC
AGTAGCCGATATGTTGGTCAGCGTCTCCAACGCGTCTGAGACCATCGTC
ATAGCGCTCATTAACGGAGGCAACCTGAGCATTCCTGTCAGGCTCATCA
AGAGCATGGACAATGTGTTTGACTCCATGATCTGCAGCTCTCTGCTGGC
CTCCATCTGCAGCTTGCTGGCCATTGCCGTTGACCGCTACATCACCATC
TTCTACGCTCTGCGATACCACAACATCGTGACGCTGCGGCGAGCAGCCG
TGGTCATCAGCAGCATCTGGACGTGCTGCATTGTGTCGGGTATCCTCTT
CATCATCTACTCGGAGAGTACCACGGTGCTCATCTGTCTCATCACCATG
TTCTTCACCATGCTGGTGCTCATGGCCTCCCTCTATGTCCACATGTTCC
TGCTGGCACGTCTGCACATGAAGCGGATCGCGGCGCTGCCGGGCAACGC
GCCCATCCACCAGCGGGCGAACATGAAGGGCGCCATCACCCTCACCATC
CTCCTCGGGGTGTTTGTGGTGTGCTGGGCGCCGTTCTTCCTCCACCTCA
TCCTCATGATCACCTGCCCCAGGAACCCTTACTGCACCTGCTTCATGTC
GCACTTCAACATGTACCTCATTCTCATCATGTGCAACTCCGTCATCGAC
CCCATCATCTACGCTTTCCGGAGCCAGGAGATGAGGAAAACCTTCAAGG
AGATCTTCTGCTGCTCCAACGCTCTCCTGTGTGTGTGA-3′

In the present invention, “loss of function” means, for example, a state in which the inherent function of the gene is deteriorated or lost. The loss of function of the MC4R gene also means, for example, a state in which the function of the MC4R gene is deteriorated or lost to such an extent that the growth of the fish according to the present invention is promoted as compared with the wild-type fish. The loss of function of the MC4R gene also means, for example, a reduction in signaling through the MC4R or a defect in the signaling. The loss of function of the MC4R gene may specifically mean, for example, a state in which the expression level of the mRNA or MC4R protein of the MC4R gene is decreased or a state in which the mRNA or MC4R protein of the MC4R gene is not fully expressed, or a state in which the expression level of the mRNA or MC4R protein of the functional MC4R gene is decreased or a state in which the mRNA or MC4R protein of the functional MC4R gene is not fully expressed.

The fish has, for example, the MC4R gene in each of a pair of autosomes. The growth promotion trait of fish obtained by the loss of function of the MC4R gene is, for example, a dominant trait. Thus, the fish according to the present invention may have, for example, loss of function of the MC4R gene in either one of the pair of autosomes or in both of the autosomes.

The loss of function of the MC4R gene can be made by mutating the normal MC4R gene, to have a mutation, more specifically a loss-of-function mutation, for example. The type of the mutation is not particularly limited, and examples thereof include point mutation, missense mutation, nonsense mutation, frameshift mutation, and extensive base deletion (large deletion). As a specific example, the loss of function of the MC4R gene can be made, for example, by mutating the normal MC4R gene such as inserting, deleting, and/or substituting one or more genes of the base sequence of the normal MC4R gene. The position of the mutation in the MC4R gene is not particularly limited, and may be any region related to the normal MC4R gene, and examples thereof include an expression control region such as a promoter region of the normal MC4R gene, a transmembrane region of the MC4R protein, a coding region encoding a MC4R protein such as a ligand-binding region, and a non-coding region not encoding the MC4R protein (for example, intron region, an enhancer region, and the like). The loss of function of the MC4R gene can promote the growth of the fish, for example. Thus, the loss of function is made by a partial or complete deletion of the MC4R gene in a preferred embodiment. The partial deletion means, for example, a partial deletion of the base sequence of the MC4R gene. The position of the partial deletion in the MC4R gene is not particularly limited and can be described with reference to the description of the position of the mutation in the MC4R gene, for example. The complete deletion means, for example, a deletion of the whole base sequence in the MC4R gene, i.e., the absence of gene encoding the MC4R protein. In this instance, the fish according to the present invention has, for example, a complete deletion of the MC4R gene.

The loss of function of the MC4R gene can be made, for example, by mutating the MC4R gene in the genomes of the target fish in a conventional manner. The mutating can be performed by, for example, genomic editing techniques using homologous recombination, ZFN, TALEN, CRISPR-CAS9, CRISPR-CPF1, or the like. The mutating may be performed by, for example, a mutagenesis method such as a site-directed mutagenesis method. The mutating may be performed also by, for example, a random mutagenesis method. The random mutagenesis method can be performed by, for example, irradiation with a radial ray such as an α-ray, a β-ray, a γ-ray, and an X-ray, treatment with a mutagen such as ethyl methanesulfonate (EMS) and ethynyl nitrosourea (ENU), and heavy ion beam. The above-mentioned mutating may be performed using, for example, a commercially available kit or the like.

The fish according to the present invention can also be produced by, for example, a production method, a screening method, and a growth promotion method according to the present invention, to be described later.

<Method for Producing Fish>

The method for producing fish according to the present invention includes interbreeding the fish according to the present invention with another fish as mentioned above. The production method according to the present invention is characterized by using the fish according to the present invention in the interbreeding, and other steps and conditions are not particularly limited. The production method according to the present invention can produce fish with promoted growth as compared to the wild-type fish, for example. The MC4R gene with loss-of-function mutation can be inherited by the progeny fish through germ cells of the fish according to the present invention. The production method according to the present invention can easily produce a MC4R gene having the loss-of-function mutation and progeny fish that inherit the trait caused by the MC4R gene having the loss-of-function mutation by interbreeding the fish according to the present invention and another fish, for example. The production method according to the present invention can be described with reference to the descriptions of the fish according to the present invention and a screening method, a growth promotion method, and a mutated MC4R gene, according to the present invention, to be described later, for example.

The fish to be used as a first parent in the interbreeding is the fish according to the present invention. As mentioned above, the fish according to the present invention can be obtained by, for example, the screening method and the growth promotion method, according to the present invention, to be described later. The fish according to the present invention can be prepared by selecting from test fish prior to the interbreeding, for example. The fish according to the present invention can be prepared by mutating the MC4R gene of target fish to have a loss-of-function mutation, for example.

In the case of selecting from the target fish, the production method according to the present invention further includes selecting the fish according to the present invention from the test fish, for example. In the selecting, the selection of the fish according to the present invention can also be referred to as selection of the test fish with loss of function of the MC4R gene. The loss of function of the MC4R gene may be identified, for example, by decoding the base sequence of the MC4R gene of the test fish and comparing it with the base sequence of the normal MC4R gene. Then, for example, if the base sequence of the MC4R gene of the test fish has a loss-of-function mutation with respect to the base sequence of the normal MC4R gene, the test fish is selected as the fish according to the present invention. The comparison of the base sequences can be performed by, for example, known base sequence analysis software. If the loss of function of the MC4R gene is made by mutating the normal MC4R gene such as inserting, deleting, and/or substituting of one or more bases of the base sequence of the normal MC4R gene, for example, the comparison may be made using a primer set, a probe, or a combination thereof, which can detect at least one mutation. The primer sets and probe can be designed using known design methods on the basis of the type of the mutation, for example. The loss of function of the MC4R gene may be identified, for example, by determining the loss of function on the basis of the function of the mRNA of the MC4R gene or the function of the MC4R protein in the test fish. Further, the loss of function of the MC4R gene may be identified by, for example, determining the loss of function on the basis of the presence or absence of expression of MC4R or the expression level of MC4R in the test fish.

If the loss of function is determined on the basis of the expression level of the MC4R, the production method according to the present invention includes, for example, measuring the expression level of MC4R in a biological sample of the test fish, and in the selecting, a test fish with loss of function of the MC4R gene is selected on the basis of the expression level of the MC4R in the biological sample of the test fish and a reference value. Specifically, the selection of the test fish with loss of function can be performed by, for example, comparing the expression level of the MC4R in the biological sample of the test fish with the reference value.

The biological sample of the test fish is not particularly limited, and may be, for example, any organ of the whole body of the test fish or a cell derived from the organ. The biological sample of the test fish may be, for example, a fin or the brain. The type of the biological sample to be used in the measuring may be, for example, one type or two or more types.

The expression level of MC4R to be measured in the measuring can be, for example, an expression level of the MC4R protein. The expression level of the MC4R protein can be measured by, for example, a method using a spectrophotometer such as an ultraviolet absorption method or a bicinchoninic acid method, or a known quantitative determination method of a protein such as ELISA or Western blotting.

Examples of the reference value include the expression level of MC4R in the wild-type fish and the expression level of MC4R in fish with loss of function of the MC4R gene (for example, fish with complete deletion of the MC4R gene). If the expression level of the MC4R gene in fish with loss of function is used as a reference value, the fish with loss of function may be fish with loss of function of either one of two MC4R genes on the respective chromosomes to be paired or fish with loss of function of both the MC4R genes, for example. The expression level of the MC4R as the reference value can be obtained, for example, by measuring the expression level of MC4R in a biological sample collected under the same condition in the same manner as the biological sample of the test fish. The reference value may be measured in advance, or may be measured at the same time as the biological sample of the test fish, for example.

In this instance, how to evaluate the loss of function of the MC4R gene in the test fish in the selecting is not particularly limited and can be appropriately determined depending on the type of the reference value. As a specific example, if the expression level of MC4R in the biological sample of the test fish is lower than the expression level of MC4R in the wild-type fish, the same as the expression level of MC4R in the fish with loss of function (with no significant difference), and/or is lower than the expression level of MC4R in the fish with loss of function, the test fish can be evaluated as having loss of function of the MC4R gene. Then, the test fish evaluated as having loss of function of the MC4R gene is selected as the fish according to the present invention, for example.

If the MC4R gene of the target fish is mutated to have a loss-of-function mutation, the production method according to the present invention further includes creating fish according to the present invention from the target fish prior to the interbreeding, for example. The creating includes mutating the MC4R gene of the target fish to have a loss-of-function mutation, for example. The loss-of-function mutation of the MC4R gene can be made, for example, by mutating the normal MC4R gene. As a specific example, the loss-of-function mutation of the MC4R gene can be made, for example, by mutating the normal MC4R gene such as inserting, deleting, and/or substituting one or more genes of the base sequence of the normal MC4R gene. In the mutating, the loss-of-function mutation is, for example, a partial or complete deletion mutation of the MC4R gene. The target fish can be, for example, the wild-type fish. The type, position, and the like of the mutation can be described with reference to the descriptions of those in the fish according to the present invention.

The mutating includes a mutation step of mutating an MC4R gene of the target fish, and a mutation selection step of selecting the target fish with a loss of function mutation of the MC4R gene from the target fish obtained in the mutation step in a preferred embodiment. How to mutate the MC4R gene to have a loss-of-function mutation in the mutating and how to perform the mutation in the mutation step can be described with reference to the description of how to perform mutating described above, for example.

In the mutation selection step, the presence or absence of the loss-of-function mutation in the MC4R gene can be determined, for example, by evaluating whether the function of the MC4R gene is lost. The loss of function of the MC4R gene may be performed, for example, by decoding the base sequence of the MC4R gene of the target fish and comparing it with the base sequence of the normal MC4R gene. The loss of function of the MC4R gene may be identified , for example, by determining the loss of function on the basis of the function of the mRNA of the MC4R gene or the function of the MC4R protein in the target fish. Further, the loss of function of the MC4R gene may be identified, for example, by determining the loss of function on the basis of the presence or absence of expression of MC4R or the expression level of MC4R in the target fish.

If the loss of function is determined on the basis of the expression level of the MC4R, the production method according to the present invention further includes, for example, a measuring step of measuring an expression level of the MC4R in a biological sample of the target fish after the mutation step, and in the mutation selection step, a target fish with loss of function of the MC4R gene is selected on the basis of an expression level of MC4R in the biological sample of the target fish and a reference value. The measuring step and the mutation selection step can be performed, for example, in the same manner as the measuring and the selecting in the case of the selection from the test fish, and can be described with reference to the descriptions thereof with replacement of the “test fish” with the “target fish” and the “selecting” with the “mutation selection step.” After the evaluation of the loss of function, a target fish evaluated as having loss of function of the MC4R gene is selected as target fish having a loss-of-function mutation of the MC4R gene of the target fish, i.e., fish according to the present invention, for example.

The interbreeding is interbreeding of the fish according to the present invention with another fish as described above. The fish according to the present invention to be used in the interbreeding is, for example, the same type of fish as the fish to be interbred therewith. The fish to be interbred with the fish according to the present invention may be a fish which is capable of being interbred with the fish according to the present invention and may be, for example, a fish having the normal MC4R gene or fish having a MC4R gene with loss of function. “Being capable of being interbred” means, for example, being capable of being naturally or artificially interbred. In the breeding, how to interbreed the fish according to the present invention with another fish is not particularly limited and can be natural breeding of male fish and female fish or artificial breeding using the gametes (sperm) of male fish and the gametes (eggs) of female fish. If the interbreeding is performed by artificial breeding, gametes are collected from the sexually mature fish according to the present invention and sexually mature another fish, for example. Then, for example, eggs derived from one fish and sperm derived from another fish are fertilized to interbreed, that is, fertilized eggs are produced. In the interbreeding, for example, germ cells or the like collected from the fish with loss of function of the MC4R gene may be transplanted to different fish, and the transplanted fish and another fish may be interbred to obtain fish with loss of function of the MC4R gene.

The production method according to the present invention may further include, for example, growing fish obtained in the interbreeding. The growing condition of and how to grow the fish can be appropriately determined according to, for example, the growth stage and type of the fish. In the growing, the fish may be grown to any growth stage, for example.

<Method for Screening for Fish with Promoted Growth>

The method for screening for fish with promoted growth (hereinafter, also referred to as “screening method”) according to the present invention includes selecting fish with loss of function of melanocortin-4 receptor (MC4R) gene from the test fish. The screening method according to the present invention is characterized by selecting the test fish with loss of function of the MC4R gene, and the steps and conditions of the screening method are not particularly limited. The screening method according to the present invention can easily screen for a fish with promoted growth, which is particularly suitable for aquaculture, for example. The screening method according to the present invention can also be referred to as the method for producing fish with promoted growth, for example. The screening method according to the present invention can be described with reference to the descriptions of the fish and the production method according to the present invention and a growth promotion method and a mutated MC4R gene according to the present invention, to be described later, for example.

The selecting in the screening method according to the present invention can be performed in the same manner as and can be described with reference to the description of the selecting in the production method according to the present invention, for example.

The screening method according to the present invention further includes measuring an expression level of melanocortin-4 receptor (MC4R) in a biological sample of test fish; and in the selecting, a fish with loss of function of the MC4R gene is selected on the basis of the expression level of the MC4R in the biological sample of the test fish and a reference value, for example.

In the screening method according to the present invention, the expression level of the MC4R is, for example, the expression level of protein of the MC4R gene.

In the screening method according to the present invention, the biological sample may be, for example, a fin or the brain.

<Method for Promoting Growth of Fish>

The method for promoting the growth of fish (hereinafter also referred to as “growth promotion method”) according to the present invention is characterized by including mutating a melanocortin-4 receptor (MC4R) gene of a target fish to have a loss-of-function mutation, as described above. The growth promotion method according to the present invention is characterized by mutating the MC4R gene to have a loss-of-function mutation, and the step and conditions are not particularly limited. The growth promotion method according to the present invention can promote the growth of the target fish, for example. The screening method according to the present invention can also be referred to as the method for producing fish with promoted growth, for example. The growth promotion method according to the present invention can be described with reference to the descriptions of the fish, production method, and screening method according to the present invention and the mutated MC4R gene according to the present invention, to be described later, for example.

The mutating in the growth promotion method according to the present invention can be performed in the same manner as and described with reference to the description of the mutating in the production method according to the present invention, for example.

In the growth promotion method according to the present invention, the loss-of-function mutation is, for example, a partial or complete deletion mutation of the MC4R gene.

In the growth promotion method according to the present invention, the mutating includes, for example, a mutation step of mutating an MC4R gene of the target fish, and a mutation selection step of selecting the target fish with a loss of function mutation of the MC4R gene from the target fish obtained in the mutation step.

The growth promotion method according to the present invention further includes, for example, a measuring step of measuring the expression level of MC4R in a of the target fish after the mutation step, and in the mutation selection step, a target fish with a loss-of-function mutation of the MC4R gene is selected on the basis of the expression level of the MC4R in the biological sample of the target fish and a reference value.

In the growth promotion method according to the present invention, the expression level of the MC4R is, for example, the expression level of the protein of the MC4R gene.

In the growth promotion method according to the present invention, the biological sample may be, for example, a fin or the brain.

<Mutated Melanocortin-4 Receptor Gene of Fish>

The mutated melanocortin-4 receptor (MC4R) gene of the fish (hereinafter also referred to as “mutated MC4R gene”) according to the present invention has a loss-of-function mutation of the MC4R gene in the fish as described above. The mutated MC4R gene according to the present invention is characterized by having a loss-of-function mutation of the MC4R gene, and the configuration and conditions thereof are not particularly limited. The mutated MC4R gene according to the present invention can promote the growth of the fish, for example. The mutated MC4R gene according to the present invention can be described with reference to the descriptions of the fish, the production method, the screening method, and the growth promotion method, according to the present invention, for example.

The MC4R gene of the fish is a normal MC4R gene of the fish. The type of the loss-of-function mutation is not particularly limited and can be described with reference to the description of the mutation described above, for example. In the mutated MC4R gene according to the present invention, the loss-of-function mutation is, for example, a partial deletion mutation of the MC4R gene.

The mutated MC4R gene according to the present invention can be produced by mutating the normal MC4R gene to have a loss-of-function mutation, for example. The mutation of the normal MC4R gene to have a loss-of-function mutation can be performed, for example, by the method to perform mutating described above.

### EXAMPLES

The present invention is described in more detail below with reference to the examples. The present invention, however, is not limited by the examples of the present invention.

### Example 1

Takifugu rubripes with loss of function of the MC4R gene were created, and it was confirmed that the growth of the Takifugu rubripes were promoted as compared to the wild-type Takifugu rubripes.

(1) Production of Fertilized Eggs

Sex hormone treatment was performed by intramuscular administration of sex hormone (females: des Gly10 [D-Ala6]-LHRH, 400 μg/kg body weight, males: des Gly10 [D-Ala6]-LHRH, 200 μg/kg body weight, or Human chorionic gonadotropin, 500 IU/kg body weight) to sexually mature male and female Takifugu rubripes. Eggs (unfertilized eggs) and sperm were collected from the male and female Takifugu rubripes, respectively, by compressing the abdomen 3 to 6 days after the sex hormone treatment.

(2) Creation of Takifugu rubripes with Loss of Function

Mutation was performed using CRISPR-Cas9 with reference to Reference 1 below. First, a Cas9 expression vector (pCS2+hSpCas9) for in vitro transcription of SP6 was prepared by the following procedures. A DNA sequence encoding Cas9 nuclease of a human codon-optimized S. pyogenes was amplified by PCR using the following primer set for Cas 9 and pX330 (Addgene Plasmid 42230). The resulting PCR product was cloned into a restriction-enzyme site (BamHI/XbaI) of a pCS2+MT vector to obtain a Cas9 expression vector. The Cas9 expression vector is available from Addgene (http://www.addgene.org).
Reference 1: Satoshi Ansai et al., “Targeted mutagenesis using CRISPR/Cas system in medaka”, Biology Open, 2014, vol. 3, pages 362-371
Reference 2: Turner, D. L. and Weintraub, H. “Expression of achaete-scute homolog 3 in Xenopus embryos converts ectodermal cells to a neural fate.”, 1994, Genes Dev, vol. 8, pages 1434-1447.

Primer set for CAS9
Forward primer
((hSpCas9FW, SEQ ID NO: 5)
5′-GCAGGATCCGCCACCATGGACTATAAGGAC-3′
Reverse primer
(hSpCas9RV, SEQ ID NO: 6)
5′-AGTTCTAGATTACTTTTTCTTTTTTGCCTGGC-3′

An expression vector of single guide RNA (sgRNA) was produced in the manner described below. In the production of the sgRNA expression vector, a pDR274 vector (Addgene Plasmid 42250) in which a T7 promoter is disposed upstream of a partial guide RNA sequence was used. A pair of oligo DNAs for each sgRNA production was synthesized and annealed, and then inserted into the pDR274 vector. After synthesizing sgRNA1 and sgRNA2 using T7-RNA polymerase, the sgRNA1 and sgRNA2 were purified using an RNA-purification cartridge. The synthesis of the pair of oligo DNAs for each sgRNA production was performed by Operon Biotechnologies. Target sites on the genomes of the sgRNA1 and sgRNA2 are shown in Table 2 below. In the base sequence of SEQ ID NO: 1, the first underlined base sequence is a target sequence of the sgRNA1, and the second underlined base sequence is a base sequence complementary to the target sequence of the sgRNA2.

TABLE 2

| sgRNA | Target sequence | Oligo DNA | Oligo DNA sequence |
|---|---|---|---|
| sgRNA1 | 5′-CAGCAACGGGAGCCAAACC | sgRNA-FuguMC4R-1S | 5′-TAGGGCAACGGGAGCCAAACCC-3′ |
| | CCGG-3′ (SEQ ID NO: 7) | | (SEQ ID NO: 8) |
| | | sgRNA-FuguMC4R-1AS | 5′-AAACGGGTTTGGCTCCCGTTGC-3′ |
| | | | (SEQ ID NO: 9) |
| sgRNA2 | 5′-CATGCTCTTGATCAGCGTG | sgRNA-FuguMC4R-2S | 5′-TAGGTGCTCTTGATCAGCGTGG-3′ |
| | GCGG-3′ (SEQ ID NO: 10) | | (SEQ ID NO: 11) |
| | | sgRNA-FuguMC4R-2AS | 5′-AAACCCACGCTGATCAAGAGCA-3′ |
| | | | (SEQ ID NO: 12) |

The pair (1S and 1AS) of oligo DNAs for sgRNA1 production was added to an annealing buffer to anneal the sgRNA1 and the complementary strand thereto so that the respective final concentrations became 10 mmol/L. The annealing buffer was composed of 40 mmol/1 L of Tris-HCl (pH8.0, 20 mmol/1 L of MgCl₂ and 50 mmol/1 L of NaCl. The annealing was performed by heat-treating at 95° C. for 2 minutes and then slowly cooling to 25° C. over 1 hour. The sgRNA2 was annealed in the same manner. The pDR274 vector was treated with a restriction enzyme (BsaI-HF, New England Biolabs) and ligated with each of sgRNA1 and sgRNA2 after the annealing, to obtain a sgRNA expression vector.

The Cas9 expression vector was then linearized by a restriction enzyme (NotI). A cap RNA (Cas9RNA) encoding Cas9 was then synthesized using the linearized Cas9 expression vector and an RNA synthesis kit (mMessage mMachine SP6 Kit, Life Technologies). The resulting cap RNA was purified using an RNA purification kit (Rneasy Mini Kit, Qiagen).

The sgRNA expression vector was linearized by a restriction enzyme (DraI). The sgRNA1 and sgRNA2 were then synthesized using the linearized sgRNA expression vector and an RNA synthesis kit (AmpliScribe T7-Flash™ Transcription Kit, Epicentre). The resulting sgRNA1 and sgRNA2 were each purified using an RNA purification kit (Rneasy Mini Kit, Qiagen).

MC4R genes were mutated by introducing 2 to 10 pg of Cas9RNA and 1 to 5 pg of sgRNA1 or sgRNA2 into the cytoplasm of the fertilized eggs at the first cell stage obtained in the above (1) by the microinjection method. As a result, individuals (Takifugu rubripes lines 1 to 3) each with a deletion of underlined 5, 7, or 13 bass in the base sequence (target sequence of sgRNA1) of any of SEQ ID Nos: 13 to 15 shown below and individuals (Takifugu rubripes lines 4 and 5) each with a deletion of underlined 4 or 5 bases in the base sequence (target sequence of sgRNA2) of SEQ ID Nos: 16 or 17, i.e., individuals with a loss-of-function mutation, were obtained.

Takifugu rubripes line 1
(SEQ ID NO: 13)
5′-CAGCAACGGGAGCCAAACCCCGG-3′
Takifugu rubripes line 2
(SEQ ID NO: 14)
5′-CAGCAACGGGAGCCAAACCCCGG-3′
Takifugu rubripes line 3
(SEQ ID NO: 15)
5′-CAGCAACGGGAGCCAAACCCCGG-3′
Takifugu rubripes line 4
(SEQ ID NO: 16)
5′-CATGCTCTTGATCAGCGTGGCGG-3
Takifugu rubripes line 5
(SEQ ID NO: 17)
5′-CATGCTCTTGATCAGCGTGGCGG-3′

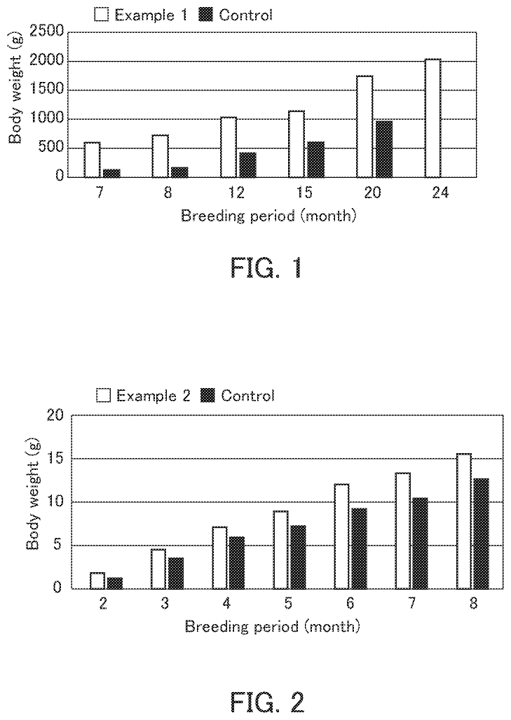

The fertilized eggs after the mutation were cultured, hatched, and then bred by a commonly used aquaculture method. The body weight of each individual (n=49, 49, 48, 48, 45, 17) was measured at 7, 8, 12, 15, 20, and 24 months after the start of the breeding. The body weight of each control individual obtained in the same manner as described above except that it is untreated was measured (n=30 for each month). The control individual was bred by Higashimal Co., Ltd. The results are shown in Table 3 below and FIG. 1.

TABLE 3

| | 7 | 8 | 12 | 15 | 20 | 24 |
|---|---|---|---|---|---|---|
| | months | months | months | months | months | months |
| Example 1 | 599.1 | 723.4 | 1031.9 | 1142.8 | 1746.3 | 2033 |
| Control | 126 | 165 | 415 | 605 | 960 | — |

(Unit of body weight: g)

FIG. 1 is a graph showing the body weight of Takifugu rubripes with loss of function of an MC4R gene. In FIG. 1, the horizontal axis represents the breeding period, and the vertical axis represents the body weight. As shown in FIG. 1 and Table 3 above, in all the breeding periods, the Takifugu rubripes of Example 1 had significantly increased body weights compared to the control Takifugu rubripes (wild-type Takifugu rubripes). The body weight of the wild-type Takifugu rubripes shipped as food is known to be 1 kg. As can be seen from Table 3 above and FIG. 1, it took 20 months or more of the breeding period for Takifugu rubripes of the control to reach a body weight of 1 kg. In contrast, the Takifugu rubripes of Example 1 reached a body weight of 1 kg at 12 months. That is, Takifugu rubripes of Example 1 grew at about twice the growth rate of Takifugu rubripes of the control.

As described above, the growth of the fish according to the present invention is promoted as compared to the wild-type fish. This demonstrates that the breeding period of the fish according to the present invention can be shortened, for example.

### Example 2

Takifugu niphobles with loss of function of the MC4R gene were created, and it was confirmed that the growth of the Takifugu niphobles were promoted as compared to wild-type Takifugu niphobles.

MC4R genes were mutated in the same manner as described above except that male and female Takifugu niphobles were used as substitute for male and female Takifugu rubripes, and the sgRNA2 is only used. The results showed that in the following sequence (targeted sequence of sgRNA1), individuals each with a deletion of underlined 5 or 7 bases (Takifugu niphobles lines 1 and 2), i.e., individuals with a loss-of-function mutation, were obtained. The underlined base sequence in the base sequence of SEQ ID NO: 3 is a base sequence complementary to the target sequence of the sgRNA2.

Takifugu niphobles line 1
(SEQ ID NO: 16)
5′-CATGCTCTTGATCAGCGTGGCGG-3′
Takifugu niphobles line 2
(SEQ ID NO: 17)
5′-CATGCTCTTGATCAGCGTGGCGG-3′

Then, the body weight of each individual (n=17) was measured at 1, 2, 3, 4, 5, 6, 7, and 8 months after the start of breeding in the same manner as in Example 1, except that the fertilized eggs of Takifugu niphobles after the mutation were used. The body weights of control individuals obtained in the same manner as described above except that they are untreated were measured (n=7 for each month). The results are shown in Table 4 below and FIG. 2.

TABLE 4

| | 2 | 3 | 4 | 5 | 6 | 7 | 8 |
|---|---|---|---|---|---|---|---|
| | month | month | month | month | month | month | month |
| Example 2 | 1.8 | 4.5 | 7.1 | 8.9 | 12 | 13.3 | 15.5 |
| Control | 1.2 | 3.5 | 5.9 | 7.2 | 9.2 | 10.4 | 12.6 |

(Unit of body weight: g)

FIG. 2 is a graph showing the body weight of Takifugu niphobles with loss of function of an MC4R gene. In FIG. 2, the horizontal axis represents the breeding period, and the vertical axis represents the body weight. As shown in FIG. 2 and Table 4 above, in all the breeding periods, the Takifugu niphobles of Example 2 had significantly increased body weights compared to the control Takifugu niphobles (wild-type Takifugu niphobles). In addition, at the breeding period of 8 months, the Takifugu niphobles of Example 2 showed 1.23 times in body weight as compared to the wild-type Takifugu niphobles, thus, the Takifugu niphobles of Example 2 showed 1.23 times in growth rate as compared to the wild-type Takifugu niphobles.

As described above, the growth of the fish according to the present invention is promoted as compared to the wild-type fish. This demonstrates that breeding period of the fish according to the present invention can be shortened, for example.

### Example 3

It was confirmed that sexual maturation of Takifugu rubripes and Takifugu niphobles with loss of function of the MC4R gene were promoted.

The female Takifugu rubripes (n=3) with loss of function of the MC4R gene obtained in Example 1 were subjected to the above-described sex hormone treatment for a breeding period of 24 months. Then, the abdomen of each female Takifugu rubripes was compressed after three to six days from the sex hormone treatment to check ovulation, and it was confirmed that all the female Takifugu rubripes had ovulated. It is generally known that sexual maturation of female wild-type Takifugu rubripes takes at least three years. In contrast, the sexual maturation of female Takifugu rubripes with loss of function of the MC4R gene took only two years and was demonstrated to be promoted.

Next, spermiation of the male Takifugu niphobles obtained in Example 2 was checked. As a result, spermiation of male Takifugu niphobles was confirmed at 6 months of the breeding period, spermiation of four individuals was confirmed at 7 months, and spermiation of five individuals was confirmed at 8 months (a total of 19 individuals of a mixture of males and females). It is generally known that sexual maturation of male wild-type Takifugu niphobles takes at least one year. In contrast, the sexual maturation of male Takifugu niphobles with loss of function of the MC4R gene took 6 months at the earliest and was demonstrated to be promoted.

This demonstrates that the sexual maturation of the fish according to the present invention was promoted as compared to the wild-type fish.

### Example 4

Oryzias latipes (Medaka)with loss of function of the MC4R gene was created, and it was confirmed that the growth of the Oryzias latipes was promoted as compared to the wild-type Oryzias latipes.

The TALEN was prepared in the manner described in Reference 3 below. That is, plasmids for synthesizing two types of TALEN (Left arm and Right arm) RNAs that specifically bind to the respective target sequences were generated by the golden gate method. This plasmid has an SP6 primer. TALEN-RNA was synthesized using the above-described RNA synthesis kit (mMessage mMachine SP6 kit, Ambion/Life Technologies) and purified by an RNA purifying column (spin column of Qiagen Rneasy mini (manufactured by Qiagen).
Reference 3: Satoshi Ansai et al., “Efficient Targeted Mutagenesis in Medaka Using Custom-Designed Transcription Activator-Like Effector Nucleases”, 2013, vol. 193, No. 3, pages 739-749

TABLE 5

| TALEN | Target sequence | Oligo DNA name | Oligo DNA sequence |
|---|---|---|---|
| TALEN1 | 5′-TCCACTCTGCCTTATGGGTCGGTCC | TALEN Left arm | 5′-TCCACTCTGCCTTATGG-3′ |
| | CCAACAGAAGCCTCTCTTCGGCCA-3′ | Binding sequence 1 | (SEQ ID NO: 21) |
| | (SEQ ID NO: 20) | TALEN Right arm | 5′-AAGCCTCTCCTCGGCCA-3′ |
| | | Binding sequence 1 | (SEQ ID NO: 22) |
| TALEN2 | 5′-TAGCCGATATGTTGGTCAGCGTCTC | TALEN Left arm | 5′-TAGCCGATATGTTGGTC-3′ |
| | CAACGCGTCTGAGACCATCGTCATA-3′ | Binding sequence 2 | (SEQ ID NO: 24) |
| | (SEQ ID NO: 23) | TALEN Right arm | 5′-CTGAGACCATCGTCATA-3′ |
| | | Binding sequence 2 | (SEQ ID NO: 25) |

The obtained TALEN-RNA was transferred into 171 fertilized eggs of Oryzias latipes by the microinjection method. The amount of RNA transferred was 5 to 15 pg. The obtained fertilized eggs were reared until they became adult fish. Among the second generation (F2 generation) individuals obtained from the adult fish, base sequences of MC4R genes of 144 individuals were decoded. As a result, individuals with a deletion of underlined 7 bases in the target sequence of the TALEN 1 and individuals with a deletion of underlined 11 basses in the target sequence of TALEN 2, i.e., individuals with a loss-of-function mutation, were obtained. The progeny obtained from these individuals were passaged to establish Oryzias latipes lines with loss of function of the MC4R gene. Hereinafter, the progeny of individuals with a deletion of 7 bases is also referred to as a Oryzias latipes line 1(Medaka line 1), and the progeny of individuals with a deletion of 11 bases is also referred to as a Oryzias latipes line 2(Medaka line 2).

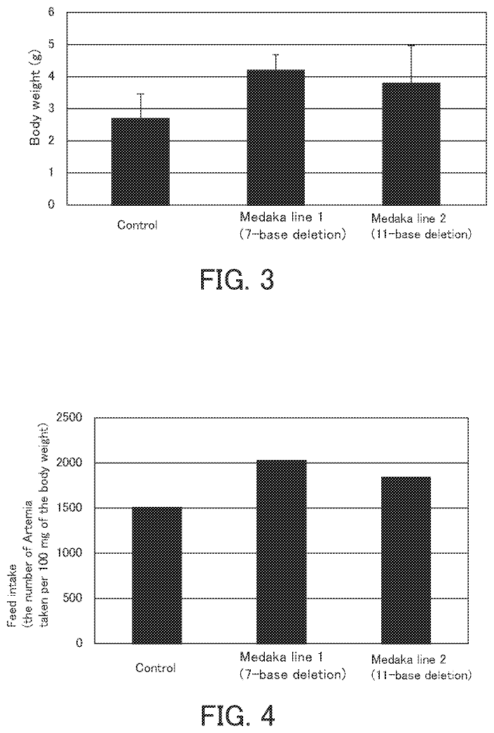

Next, the body weight of each of ten individuals of Oryzias latipes derived from the Oryzias latipes lines 1 and 2 were measured at 2 weeks after hatching. The body weight of each control individual was also measured in the same manner except that they are untreated. The results are shown in Table 6 below and FIG. 3.

TABLE 6

| | | Oryzias latipes | Oryzias latipes |
|---|---|---|---|
| | | line 1 | line 2 |
| | Control | (7-base deletion) | (11-base deletion) |
| Body weight | 2.68 | 4.19 | 3.79 |
| (g) | | | |

FIG. 3 is a graph showing the body weight of Oryzias latipes with loss of function of an MC4R gene. In FIG. 3, the horizontal axis represents the type of Oryzias latipes, and the vertical axis represents the body weight. As shown in FIG. 3 and Table 6 above, Oryzias latipes of Example 4 had a significant increase in body weight compared to the control Oryzias latipes (wild-type Oryzias latipes).

As described above, the growth of the fish according to the present invention is promoted as compared to the wild-type fish. This demonstrates that breeding period of the fish according to the present invention can be shortened, for example.

### Example 5 Oryzias latipes with loss of function of the MC4R gene were created, and it was confirmed that the feed intake of Oryzias latipes was increased as compared to the wild-type Oryzias latipes.

Each of six individuals of the Oryzias latipes lines 1 and 2 and the control Oryzias latipes (wild-type Oryzias latipes) were placed in water bath, and counted Artemia were fed as a feed. The number of Artemia remaining after four hours from the feeding was measured, and the number of Artemia taken by the Oryzias latipes was determined from the difference from the number of Artemia before feeding. The feed intake was calculated as the number of Artemia taken per 100 mg of the body weight of the Oryzias latipes. The same test was performed two more times, and the average of the results of the three tests was calculated. The results are shown in Table 7 below and FIG. 4.

TABLE 7

| | | Oryzias latipes | Oryzias latipes |
|---|---|---|---|
| | Wild type | line 1 | line 2 |
| Average | 1506 | 2024 | 1839 |
| Standard error | 150.6 | 166.2 | 103.6 |

FIG. 4 is a graph showing the feed intake of Oryzias latipes with loss of function of the MC4R gene. In FIG. 4, the horizontal axis represents the type of Oryzias latipes, and the vertical axis represents the feed intake. As shown in FIG. 4 and Table 7 above, the Oryzias latipes of Example 4 had significantly increased feed intake compared to the control Oryzias latipes (wild-type Oryzias latipes).

As described above, the feed intake of the fish according to the present invention is increased as compared to the wild-type fish. This demonstrates that the growth of the fish according to the present invention is promoted. However, this assumption does not at all limit the present invention.

### Example 6

It was confirmed that next-generation individuals inherited loss of function of the MC4R gene through germ cells, and the growth of the next-generation individuals was promoted.

Takifugu rubripes of the Takifugu rubripes line 3 of Example 1 (three males, three females) were bred until sexual maturation. Sperm or unfertilized eggs were collected from each individual after sexual maturation, and artificial fertilization was performed with unfertilized eggs or sperm collected from the wild-type Takifugu rubripes. The resulting fertilized eggs were cultured at 20° C. for five days. Genomic DNA was extracted from the obtained embryos, and the base sequence of the MC4R gene in the genomic DNA was analyzed to determine whether the genomic DNA of the germ cells of each individual of Takifugu rubripes inherited the loss-of-function mutation of the MC4R gene. This result showed that 20% of germ cells in one individual among three males had a mutation of a deletion of underlined 13 bases in SEQ ID NO: 15. This result also showed that 18% of germ cells in one individual among three females had a mutation of a deletion of underlined 13 bases in SEQ ID NO: 15.

Next, male and female individuals whose germ cells inherited the loss-of-function mutation of the MC4R gene were interbred to obtain a second generation (F2 generation). The resulting 50 F2-generation individuals were bred for 14 months. Genomic DNA was extracted from each individual after the breeding, and the base sequence of the MC4R gene in the genomic DNA was analyzed. The result showed that 10% of the F2-generation individuals (five individuals) had a homozygous mutation of a deletion of underlined 13 based in SEQ ID NO: 15, i.e., had a homozygous-mutated MC4R gene. The body weight of each individual with a homozygous mutation (n=5) was measured at 14 months of the breeding. The body weight of each control individual (n=5) was measured in the same manner except that untreated Takifugu rubripes was used as in Example 1. This result showed that the body weights of the second-generation Takifugu rubripes having a homozygous-mutated MC4R gene significantly increased as compared to the control Takifugu rubripes (wild-type Takifugu rubripes).

This demonstrates that next-generation individuals inherit loss of function of the MC4R gene through germ cells, and the growth of the next-generation individuals is promoted.

While the present invention has been described above with reference to exemplary embodiments and examples, the present invention is by no means limited thereto. Various changes and modifications that may become apparent to those skilled in the art may be made in the configuration and specifics of the present invention without departing from the scope of the present invention.

This application is based upon and claims the benefit of priority from Japanese patent application No. 2017-187374, filed on Sep. 28, 2017, the disclosure of which is incorporated herein its entirety by reference.

### INDUSTRIAL APPLICABILITY

As described above, since the fish according to the present invention have loss of function of the MC4R gene, the growth of fish is promoted as compared to, for example, the wild-type fish. The fish according to the present invention allow, for example, the breeding period until the fish reaches the target growth stage to be shorten as compared to the wild-type fish. Thus, the fish according to the present invention are suitable for use as fish for aquaculture. Although the mechanism is unknown, the period of time required for the fish according to the present invention to be grown as sexually mature fish is shorter than that of the wild-type. For example. For this reason, the fish according to the present invention can be interbred (for example, egg collection, sperm collection) in a shorter period of time, as compared to the wild-type fish, for example. Thus, the fish according to the present invention are suitable for use as fish for aquaculture. Therefore, the present invention is extremely useful in the field of fishery such as aquaculture, for example.

### SEQUENCE LISTING

TF17096WO_sequence listing_ST25.txt

1. Fish with loss of function of a melanocortin-4 receptor (MC4R) gene.

2. The fish of claim 1, with a partial or complete deletion of the MC4R gene.

3. The fish of claim 1, belonging to at least one selected from the group consisting of the family Tetraodontidae, the family Sparidae, the family Serranidae, and the family Adrianichthyidae.

4. The fish of claim 1, for use in aquaculture.

5. The fish of claim 1, wherein the fish is an edible portion of the fish.

6. A method for producing fish, the method comprising: interbreeding the fish claim 1 with another fish.

7. The method of claim 6, further comprising growing the fish obtained by the interbreeding.

8. The method of claim 6, further comprising, prior to the interbreeding,
measuring an expression level of melanocortin-4 receptor (MC4R) in a biological sample of test fish;
wherein
a test fish with loss of function of the MC4R gene is selected on the basis of the expression level of the MC4R in the biological sample of the test fish and a reference value.

9. The method of claim 6, further comprising, prior to the interbreeding, creating a fish with loss of function of a melanocortin-4 receptor (MC4R) gene from a target fish, wherein
the creating includes mutating the MC4R gene of the target fish to have a loss-of-function mutation.

10. The method for promoting the growth of fish, the method comprising mutating a melanocortin-4 receptor (MC4R) gene of target fish to have a loss-of-function mutation.

11. The method of claim 10, wherein the mutating step comprising
mutating an MC4R gene of the target fish, and
selecting the target fish with a loss of function mutation of the MC4R gene from the target fish obtained in the mutation step.

12. The method of claim 10, further comprising, measuring the expression level of MC4R in a of the target fish after the mutation step, and
in the mutation step, target fish with a loss-of-function mutation of the MC4R gene is selected on the basis of the expression level of the MC4R in the biological sample of the target fish and a reference value.

13. The method of claim 10, wherein the loss-of-function mutation is a partial of complete deletion mutation of the MC4R gene.


SEQUENCE LISTING (25 sequences)

SEQ ID NO: 1 - DNA, 969 bp, Takifugu rubripes
atgaacgccaccgatccccctgggagggtgcaggacttcagcaacgggagccaaaccccggagacggactttccaaacgaggagaaggaatcgtctacgggatgctacgagcagatgctgatctccacggaggtgttcctgactctgggaatcatcagcctgctggagaacatcctggtggtcgccgctatagtgaagaacaagaatctccactcgcccatgtactttttcatctgcagcctggccgtggccgacatgctcgtgagcgtctccaacgcctccgagacgatcgtcatagcgctcatcaacagcggcacgctgaccatccccgccacgctgatcaagagcatggacaacgtgtttgactccatgatctgcagctctttgctggcgtccatctgcagcctgctcgccatcgccgtcgaccgctacatcaccatcttctacgccctgcgctaccacaacatcgtcaccctgcggagagcctcgctggtcatcagcagcatctggacgtgctgcaccgtgtccggcgtgctcttcatcgtctactcggagagcaccaccgtgctcatctgcctcatcaccatgttcttcaccatgctggtgctcatggcctccctctacgtccacatgttcctgctggcgcgcctgcacatgaagcggatcgcggcgatgccgggcaacgcgcccatccaccagagagccaacctgaagggcgccatcaccctcaccatcctcctgggagtgtttgtggtctgctgggcgcctttcttccttcacctcatcctcatgatcacctgccccaagaacccatactgcacgtgcttcatgtcccacttcaacatgtacctcatcctcatcatgtgcaactccgtcatcgaccccatcatctacgcctttcgcagccaggagatgagaaaaaccttcaaggagatcttctgctgctcccaaatgctggtgtgcatgtga

SEQ ID NO: 2 - DNA, 981 bp, Pagrus major
atgaacagcacagatctccatggattgatccagggctaccacaacaggagccaaacgtcagttttgcctctgaacaaagacttaccagccgaggagaaggactcatcggcaggatgctacgaacagctgctgatttctacagaggtgttcctcactctgggcatcatcagcctgctggagaacatcctggttgttgctgcaatcgtcaagaacaagaaccttcactcgcccatgtacttcttcatctgtagcctcgctgttgctgacatgctcgtgagcgtctccaacgcctccgagaccatcgtcatagcgctcatcgatggaggcaacctgaccatccccgccacgctgatcaagaacatggacaatgtatttgactctatgatctgtagctctctgttagcgtctatctgcagcttgctcgccatcgccatcgatcgctacatcaccatcttctacgcgctgcggtaccacaacattgtcaccctgcggagagccatattggtcatcagcagcatctggacgtgctgcaccgtctctggcatcctcttcatcatctactcagagagcaccacggtgctcatctgcctcatcaccatgttcttcaccatgctcgttctcatggcgtcgctctacgtgcacatgttccttctggcgcgcttgcacatgaagcggatcgccgctctgccgggcaacgcgcccatccaccagcgggccaacatgaagggcgccatcaccctcaccatcctcctcggggtgttcgtggtgtgctgggcgcccttcttcctccacctcatcctcatgatcacctgccccaggaacccctactgcacctgcttcatgtcccacttcaacatgtacctcatcctcatcatgtgcaactccgtcatcgaccccatcatctacgctttccgcagccaggagatgaggaagaccttcaaggagattttctgctgctctcacactttcctgtgcgtgtga

SEQ ID NO: 3 - DNA, 788 bp, Takifugu niphoble
ggaggtgttcctgactctgggaatcatcagcctgctggagaacatcctggtggtcgccgctatagtgaagaacaagaatctccactcgcccatgtactttttcatctgcagcctggccgtggccgacatgctcgtgagcgtctccaacgcctccgagacgatcgtcatagcgctcatcaacagcggcacgctcaccatccccgccacgctgatcaagagcatggacaacgtgtttgactccatgatctgcagctccttgctggcgtccatctgcagcctgctcgccatcgccgtcgaccgctacatcaccatcttctacgccctgcgctaccacaacatcgtcaccctgcggagagcctcgctggtcatcagcagcatctggacgtgctgcaccgtgtccggcgtgctcttcatcgtctactcggagagcaccaccgtgctcatctgcctcatcaccatgttcttcaccatgctggtgctcatggcctccctctacgtccacatgttcctgctggcgcgcctgcacatgaagcggatcgcggcgatgccgggcaacgcgcccatccaccagagagccaacctgaagggcgccatcaccctcaccatcctcctgggagtgtttgtggtctgctgggcgcctttcttccttcacctcatcctcatgatcacctgccccaagaacccatactgcacgtgcttcatgtcccacttcaacatgtacctcatcctcatcatgtgcaactccgtcatcgaccccatcatctacgcctttcgcagccaggagatga

SEQ ID NO: 4 - DNA, 966 bp, Oryzias latipes
atgaactccactctgccttatgggtcggtccccaacagaagcctctcctcggccactctccctcctgacctgggaggacagaaagactcgtcggcgggatgctacgagcagcttctgatctccactgaggtcttcctcactttgggcatcatcagcctgctggagaacatcctggttgttgctgcgatcgttaaaaacaagaacctccactcccccatgtactttttcatctgcagcctcgcagtagccgatatgttggtcagcgtctccaacgcgtctgagaccatcgtcatagcgctcattaacggaggcaacctgagcattcctgtcaggctcatcaagagcatggacaatgtgtttgactccatgatctgcagctctctgctggcctccatctgcagcttgctggccattgccgttgaccgctacatcaccatcttctacgctctgcgataccacaacatcgtgacgctgcggcgagcagccgtggtcatcagcagcatctggacgtgctgcattgtgtcgggtatcctcttcatcatctactcggagagtaccacggtgctcatctgtctcatcaccatgttcttcaccatgctggtgctcatggcctccctctatgtccacatgttcctgctggcacgtctgcacatgaagcggatcgcggcgctgccgggcaacgcgcccatccaccagcgggcgaacatgaagggcgccatcaccctcaccatcctcctcggggtgtttgtggtgtgctgggcgccgttcttcctccacctcatcctcatgatcacctgccccaggaacccttactgcacctgcttcatgtcgcacttcaacatgtacctcattctcatcatgtgcaactccgtcatcgaccccatcatctacgctttccggagccaggagatgaggaaaaccttcaaggagatcttctgctgctccaacgctctcctgtgtgtgtga

SEQ ID NO: 5 - DNA, 30 bp, Artificial Sequence
  Synthesized oligonucleotide(hSpCas9FW)
gcaggatccgccaccatggactataaggac

SEQ ID NO: 6 - DNA, 32 bp, Artificial Sequence
  Synthesized oligonucleotide(hSpCas9RV)
agttctagattactttttcttttttgcctggc

SEQ ID NO: 7 - DNA, 23 bp, Takifugu rubripes
cagcaacgggagccaaaccccgg

SEQ ID NO: 8 - DNA, 22 bp, Artificial Sequence
  Synthesized oligonucleotide(sgRNA-FuguMC4R-1)
tagggcaacgggagccaaaccc

SEQ ID NO: 9 - DNA, 22 bp, Artificial Sequence
  Synthesized oligonucleotide(sgRNA-FuguMC4R-1AS)
aaacgggtttggctcccgttgc

SEQ ID NO: 10 - DNA, 23 bp, Takifugu rubripes
catgctcttgatcagcgtggcgg

SEQ ID NO: 11 - DNA, 22 bp, Artificial Sequence
  Synthesized oligonucleotide(sgRNA-FuguMC4R-2S)
taggtgctcttgatcagcgtgg

SEQ ID NO: 12 - DNA, 22 bp, Artificial Sequence
  Synthesized oligonucleotide(sgRNA-FuguMC4R-2AS)
aaacccacgctgatcaagagca

SEQ ID NO: 13 - DNA, 23 bp, Takifugu rubripes
cagcaacgggagccaaaccccgg

SEQ ID NO: 14 - DNA, 23 bp, Takifugu rubripes
cagcaacgggagccaaaccccgg

SEQ ID NO: 15 - DNA, 23 bp, Takifugu rubripes
cagcaacgggagccaaaccccgg

SEQ ID NO: 16 - DNA, 23 bp, Takifugu rubripes
catgctcttgatcagcgtggcgg

SEQ ID NO: 17 - DNA, 23 bp, Takifugu rubripes
catgctcttgatcagcgtggcgg

SEQ ID NO: 18 - DNA, 23 bp, Takifugu niphoble
catgctcttgatcagcgtggcgg

SEQ ID NO: 19 - DNA, 23 bp, Takifugu niphoble
catgctcttgatcagcgtggcgg

SEQ ID NO: 20 - DNA, 49 bp, Oryzias latipes
tccactctgccttatgggtcggtccccaacagaagcctctcctcggcca

SEQ ID NO: 21 - DNA, 17 bp, Artificial Sequence
  Synthesized oligonucleotide(TALEN left arm binding sequence 1)
tccactctgccttatgg

SEQ ID NO: 22 - DNA, 17 bp, Artificial Sequence
  Synthesized oligonucleotide(TALEN right arm binding sequence 1)
aagcctctcctcggcca

SEQ ID NO: 23 - DNA, 50 bp, Oryzias latipes
tagccgatatgttggtcagcgtctccaacgcgtctgagaccatcgtcata

SEQ ID NO: 24 - DNA, 17 bp, Artificial Sequence
  Synthesized oligonucleotide(TALEN left arm binding sequence 2)
tagccgatatgttggtc

SEQ ID NO: 25 - DNA, 17 bp, Artificial Sequence
  Synthesized oligonucleotide(TALEN right arm binding sequence 2)
ctgagaccatcgtcata